(19) United States
(12) Patent Application Publication
(10) Pub. No.: US 2014/0363893 A1
(43) Pub. Date: Dec. 11, 2014

(54) DIAGNOSING METHOD FOR OIL-FILLED ELECTRICAL EQUIPMENT

(71) Applicants: Ryuichi Nishiura, Chiyoda-ku (JP); Fukutaro Kato, Chiyoda-ku (JP); Eiichi Nagao, Chiyoda-ku (JP)

(72) Inventors: Ryuichi Nishiura, Chiyoda-ku (JP); Fukutaro Kato, Chiyoda-ku (JP); Eiichi Nagao, Chiyoda-ku (JP)

(73) Assignee: MITSUBISHI ELECTRIC CORPORATION, Chiyoda-ku Tokyo (JP)

(21) Appl. No.: 14/241,336

(22) PCT Filed: Nov. 28, 2011
(86) PCT No.: PCT/JP2011/077330

§ 371 Date: Feb. 26, 2014

(51) Int. Cl. G01N 33/28

(52) U.S. Cl. CPC G01N 33/2835; G01N 33/287
USPC 436/60

(57) ABSTRACT

The present invention is a diagnosing method for oil-filled electrical equipment for diagnosing a degree of risk with regard to occurrence of abnormality due to copper sulfide generation in oil-filled electrical equipment, and the method includes a first step of detecting specific compounds contained in insulating oil in said oil-filled electrical equipment, a second step of evaluating a possibility of copper sulfide generation at a dangerous part leading to dielectric breakdown in said oil-filled electrical equipment in accordance with a detection result obtained in said first step, and a third step of diagnosing a degree of risk with regard to occurrence of abnormality in said oil-filled electrical equipment in accordance with an evaluation result obtained in said second step. Said specific compounds include dibenzyldisulfide and 2,6-di-t-butyl-p-cresol.

### TECHNICAL FIELD

The present invention relates to a diagnosing method for diagnosing a degree of risk with regard to occurrence of abnormality in an oil-filled electrical equipment by evaluating a risk of copper sulfide generation causing dielectric breakdown on insulating paper in oil-filled electrical equipment such as a transformer in which coil copper having insulating paper wound therearound is arranged in insulating oil.

### BACKGROUND ART

In oil-filled electrical equipment such as an oil-filled transformer, coil copper as a conducting medium has coil insulating paper wound therearound so that a structure for preventing occurrence of short-circuit between adjoined turn is provided.

Meanwhile, mineral oil used in the oil-filled transformer contains a sulfur constituent, and the sulfur constituent reacts with coil copper arranged in oil, so that conductive copper sulfide is generated. In the case where this copper sulfide is generated on a surface of insulating paper provided on the coil, a conduction path is formed from a point at which copper sulfide is deposited because copper sulfide is a conductive substance. Consequently, there has been known disadvantages such as occurrence of dielectric breakdown due to short-circuit of adjoining coil turns (for example, NPD 1 (CIGRE WG A2-32, “Copper sulphide in transformer insulation,” Final Report Brochure 378, 2009)).

Moreover, it has been known that a causative substance causing generation of copper sulfide is dibenzyldisulfide which is a kind of a sulfur compound in oil (for example, NPD 2 (F. Scatiggio, V. Tumiatti, R. Maina, M. Tumiatti, M. Pompilli and R. Bartnikas, “Corrosive Sulfur in Insulating Oils: Its Detection and Correlated Power Apparatus Failures”, IEEE Trans. Power Del., Vol. 23, pp. 508-509, 2008)).

It has been known that copper sulfide is generated on coil insulating paper by a process in which dibenzyldisulfide reacts with coil copper to generate a complex, a process in which the complex is diffused in oil to adhere to coil insulating paper, and a process in which the adhered complex is decomposed to become copper sulfide (for example, NPD 3 (S. Toyama, J. Tanimura, N. Yamada, E. Nagao and T. Amimoto, “Highly Sensitive Detection Method of Dibenzyl Disulfide and the Elucidation of the Mechanism of Copper Sulfide Generation in Insulating Oil”, IEEE Transactions on Dielectrics and Electrical Insulation, Vol. 16, No. 2, pp. 509-515, 2009.)).

There has been a known method for suppressing copper sulfide generation by suppressing the reaction between dibenzyldisulfide and coil copper in accordance with the generation mechanism described above, and a method of adding an inhibitor to electric insulating oil is widely used. As an inhibitor for suppressing copper sulfide generation, 1,2,3-benzotriazole (BTA) or Irgamet39 is used (for example, NPD 4 (T. Amimoto, E. Nagao, J. Tanimura, S. Toyama and N. Yamada, “Duration and Mechanism for Suppressive Effect of Triazole-based Passivators on Copper-sulfide Deposition on Insulating Paper”, IEEE Transactions on Dielectrics and Electrical Insulation, Vol. 16, No. 1, pp. 257-264, 2009.)).

When an inhibitor for copper sulfide generation is added to oil, the inhibitor reacts with coil copper to form a coat on a surface of the coil copper (for example, PTD 1 (Japanese Patent Laying-Open No. 6-76635)). Since this formed coat blocks or suppresses reaction between dibenzyldisulfide and coil copper, copper sulfide generation can be suppressed (for example, NPD 4).

Since insulating oil used in oil-filled electrical equipment such as a transformer is generally large in the amount and exhibits a long age of service, replacement is not easy. Therefore, in each oil-filled electrical equipment using insulating oil containing a sulfur constituent, a method for predicting occurrence of abnormality such as dielectric breakdown caused by deposition of copper sulfide is required.

However, the part at which copper sulfide is generated in oil-filled electrical equipment is not only on coil insulating paper, but also at coil copper, PB (press board) and the like, and the risk of occurrence of abnormality such as dielectric breakdown is different at respective parts. Therefore, there has been a disadvantage that the risk of occurrence of abnormality in oil-filled electrical equipment cannot be evaluated flatly by predicting a possibility of copper sulfide generation with mere measurement of a causative substance such as dibenzyldisulfide or the like.

### CITATION LIST

### Patent Document

PTD 1: Japanese Patent Laying-Open No. 6-76635

### NON PATENT DOCUMENT

NPD 1: CIGRE WG A2-32, “Copper sulphide in transformer insulation,” Final Report Brochure 378, 2009
NPD 2: F. Scatiggio, V. Tumiatti, R. Maina, M. Tumiatti M. Pompilli and R. Bartnikas, “Corrosive Sulfur in Insulating Oils: Its Detection and Correlated Power Apparatus Failures”, IEEE Trans. Power Del., Vol. 23, pp. 508-509, 2008
NPD 3: S. Toyama, J. Tanimura, N. Yamada, E. Nagao and T. Amimoto, “Highly Sensitive Detection Method of Dibenzyl Disulfide and the Elucidation of the Mechanism of Copper Sulfide Generation in Insulating Oil”, IEEE Transactions on Dielectrics and Electrical Insulation, Vol. 16, No. 2, pp. 509-515, 2009.
NPD 4: T. Amimoto, E. Nagao, J. Tanimura, S. Toyama and N. Yamada, “Duration and Mechanism for Suppressive Effect of Triazole-based Passivators on Copper-sulfide Deposition on Insulating Paper”, IEEE Transactions on Dielectrics and Electrical Insulation, Vol. 16, No. 1, pp. 257-264, 2009.

### SUMMARY OF INVENTION

### Technical Problem

The present invention was achieved to solve the problem described above, and its object is to provide a diagnosing method capable of diagnosing a degree of risk with regard to occurrence of abnormality (dielectric breakdown) in oil-filled electrical equipment at high accuracy by evaluating a component in insulating oil to thereby estimate a possibility of copper sulfide generation at a dangerous part leading to the dielectric breakdown.

### Solution to Problem

The diagnosing method according to the present invention evaluates a risk of copper sulfide generation at a dangerous part (surface of insulating paper) leading to dielectric break down by evaluating presence or absence of 2,6-di-t-butyl-p-cresol (DBPC) accelerating copper sulfide deposition to a surface of insulating paper in addition to conventional diagnosis items (presence or absence of dibenzyldisulfide (DBDS) and the like).

In other words, the present invention is a diagnosing method for oil-filled electrical equipment for diagnosing a degree of risk with regard to occurrence of abnormality due to copper sulfide generation in oil-filled electrical equipment, and the method includes:

a first step of detecting specific compounds contained in insulating oil in said oil-filled electrical equipment;

a second step of evaluating a possibility of copper sulfide generation at a dangerous part leading to dielectric breakdown in said oil-filled electrical equipment in accordance with a detection result obtained in said first step; and

a third step of diagnosing a degree of risk with regard to occurrence of abnormality in said oil-filled electrical equipment in accordance with an evaluation result obtained in said second step, and

said specific compounds include dibenzyldisulfide and 2,6-di-t-butyl-p-cresol.

Preferably, in the diagnosing method according to the present invention, said dangerous part is a surface of insulating paper applied to a surface of a coil winding wire.

Preferably, said specific compounds include a byproduct which is derived when copper sulfide is generated from dibenzyldisulfide. Preferably, said byproduct is at least one kind of compound selected from a group consisting of benzaldehyde, benzyl alcohol, bibenzyl, dibenzyl sulfide, and dibenzyl sulphoxide.

Preferably, said specific compounds include an inhibitor for suppressing copper sulfide generation. Preferably, said inhibitor for suppressing copper sulfide generation is a benzotriazole compound.

Preferably, in said second step, a possibility of copper sulfide generation at a dangerous part leading to dielectric breakdown in said oil-filled electrical equipment is evaluated based on whether each of said specific compounds is detected or not in said first step, and

in said third step, a degree of risk with regard to occurrence of abnormality in said oil-filled electrical equipment is diagnosed as being high when a possibility of copper sulfide generation is evaluated as being high in said second step.

Preferably, in said second step, a possibility of copper sulfide generation is evaluated taking into consideration presence or absence of oxygen in an atmosphere of said insulating oil.

Preferably, in said second step, a possibility of copper sulfide generation is evaluated further taking into consideration presence or absence of said copper sulfide generation at a moment of diagnosis.

### Advantageous Effects of Invention

According to the diagnosing method of the present invention, DBPC is added as a diagnosis item in addition to conventional diagnosis items, so that a possibility of copper sulfide generation on a surface of insulating paper of a coil or the like dipped in insulating oil in oil-filled electrical equipment can be evaluated. Accordingly, a degree of risk with regard to occurrence of abnormality in oil-filled electrical equipment can be diagnosed accurately.

### BRIEF DESCRIPTION OF DRAWINGS

FIG. 1 is a schematic diagram for describing a mechanism of copper sulfide generation on insulating paper.

FIG. 2 is a schematic diagram for describing a temporal change in the amounts of DBDS and copper sulfide generation.

### DESCRIPTION OF EMBODIMENTS

In oil-filled electrical equipment such as an oil-filled transformer, a sulfur constituent contained in insulating oil thereof reacts with a coil (copper part) to generate copper sulfide. In the case where the location of this copper sulfide generation is on insulating paper providing insulation between coil winding wires, insulation between turns is deteriorated, so that a risk of dielectric breakdown (occurrence of abnormality) becomes high. The present invention relates to a method for diagnosing a degree of risk with regard to occurrence of abnormality by evaluating copper sulfide generation on insulating paper provided in such oil-filled electrical equipment.

In oil-filled electrical equipment such as an oil-filled transformer, it is difficult to examine during operation a coil portion which may cause a problem. Therefore, a component analysis of insulating oil extracted from the oil-filled electrical equipment is conducted to evaluate a possibility of copper sulfide generation. According to the present invention, at least DBDS and DBPC present in insulating oil are used as indices to evaluate a possibility of copper sulfide generation. It should be noted that DBPC is a substance which is in some cases added as an antioxidant to insulating oil.

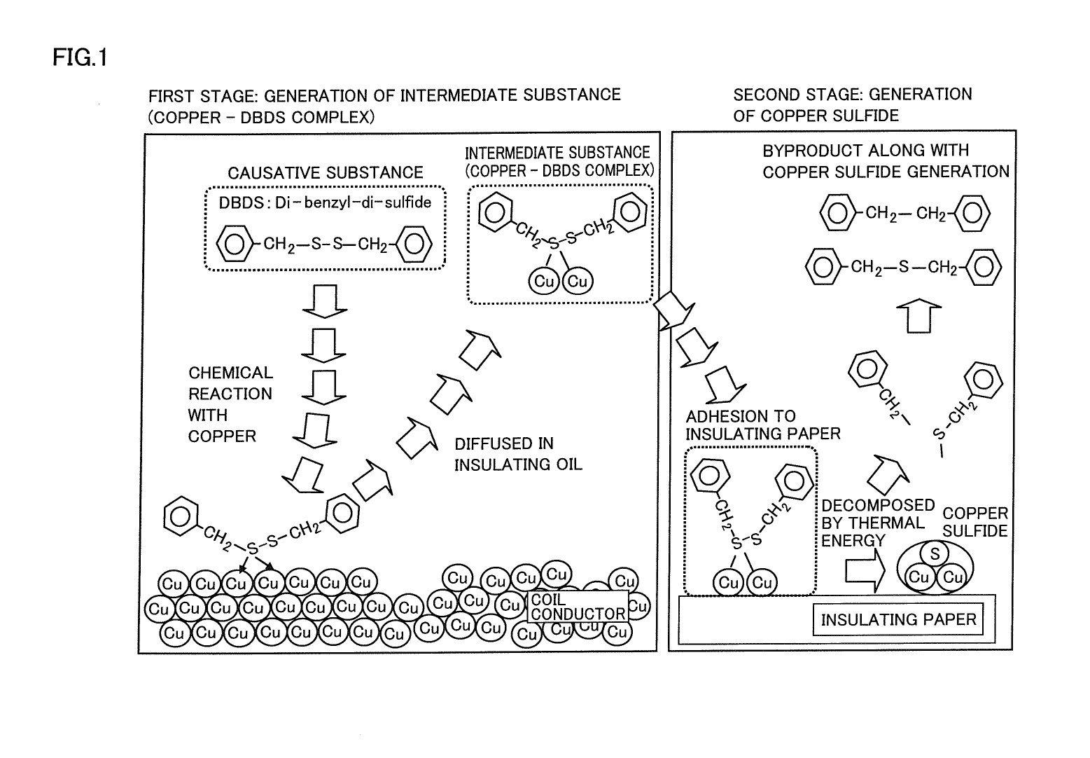

FIG. 1 shows a mechanism of generation of copper sulfide inside oil-filled electrical equipment in a state where air is not present (nitrogen atmosphere). As shown in FIG. 1, the formation reaction of copper sulfide is constituted of two stages. In the first stage, a copper-DBDS complex (intermediate substance) is generated by a chemical reaction between copper and DBDS (causative substance). This complex is diffused in insulating oil and partially adheres to insulating paper. In the second stage, the complex is decomposed by thermal energy, so that copper sulfide is deposited on the insulating paper (for example, NPD 3).

As described above, copper sulfide generation on the surface of insulating paper generally takes much time since copper sulfide reacts with dibenzyldisulfide and coil copper and subsequently proceeds through diffusion of a reaction product in oil, adhesion to the insulating paper surface, and thermal decomposition of product.

On the other hand, as a result of conducting a test as to copper sulfide generation on a surface of insulating paper based on presence or absence of DBDS and DBPC, it was found out that presence of DBPC accelerates generation of copper sulfide on the surface of insulating paper. In other words, it is considered that, as compared to the case where DBPC is not present, a risk of dielectric breakdown is very high in the case where DBPC is present. Further, it was found out that there is a case where generation of copper sulfide on the surface of insulating paper is accelerated according to the difference in atmosphere at the time of test.

Thus, according to the present invention, a possibility of copper sulfide generation on insulating paper, in other words, a risk of dielectric breakdown is evaluated based on DBPC as an index in addition to the analysis result of DBDS or the like in insulating oil. As described above, subdivision and improvement in the risk analysis can be achieved by adding the determination on presence or absence of DBPC accelerating copper sulfide generation on a dangerous part (on insulating paper) of dielectric breakdown. It should be noted that presence or absence of DBPC may be determined not only by the result of component analysis of insulating oil but also by the bland of insulating oil used.

In other words, the diagnosing method of the present invention includes (1) a first step of detecting specific compounds contained in insulating oil in said oil-filled electrical equipment, (2) a second step of evaluating a possibility of copper sulfide generation at a dangerous part leading to dielectric breakdown in said oil-filled electrical equipment in accordance with a detection result obtained in said first step, and (3) a third step of diagnosing a degree of risk with regard to occurrence of abnormality in said oil-filled electrical equipment in accordance with an evaluation result obtained in said second step.

For example, in the second step, a possibility of copper sulfide generation at a dangerous part leading to dielectric breakdown in the oil-filled electric equipment is evaluated based on whether each of said specific compounds is detected or not in first step. In the case where it is evaluated that a possibility of copper sulfide generation is high, in the next third step, it is diagnosed that a degree of risk with regard to occurrence of abnormality in the oil-filled electrical equipment is high.

The specific compounds include at least dibenzyldisulfide (DBDS) and 2,6-di-t-butyl-p-cresol (DBPC). In the first step, at least both of these are detected (measured). Further, the dangerous part leading to dielectric breakdown in oil-filled electrical equipment is, for example, a surface of insulating paper applied to the coil winding wire surface.

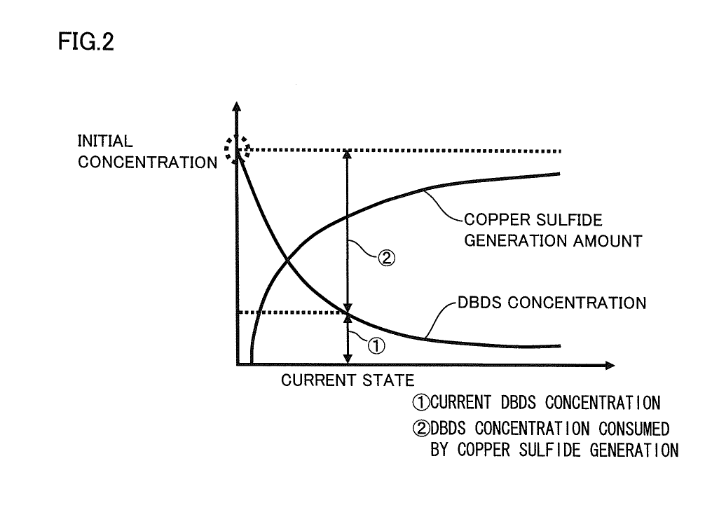

Herein, since DBDS is used and reduced as copper sulfide generation proceeds (refer to FIG. 2), there is a possibility that an erroneous evaluation is made if the possibility of copper sulfide generation is evaluated based only on the amount of DBDS. Therefore, it is preferable to evaluate a possibility of copper sulfide generation based not only on DBDS but also on a byproduct derived at the time of generation of copper sulfide from DBDS, as indices. The byproduct may include, for example, benzaldehyde, benzyl alcohol, bibenzyl, dibenzyl sulfide, and dibenzyl sulphoxide.

Further, since a possibility of copper sulfide generation differs depending on presence or absence of an inhibitor for copper sulfide generation (BTA or the like) and a difference in atmosphere of insulating oil (presence or absence of oxygen) in addition to the byproduct, it is preferable to take into consideration of those when evaluating a possibility of copper sulfide generation.

Thus, the specific compounds preferably include an inhibitor for suppressing copper sulfide generation. Preferably, the inhibitor for suppressing copper sulfide generation is a benzotriazole compound. The benzotriazole compound may include, for example, 1,2,3-benzotriazole (BTA), Irgamet (registered trademark) 39 [N,N-bis(2-ethylhexyl)-(4 or 5)-methyl-1H-benzotriazole-1-methylamine: manufactured by BASF Japan Ltd.].

Further, in the second step, it is preferable to evaluate a possibility of copper sulfide generation taking into consideration the presence or absence of oxygen in an atmosphere of insulating oil and the presence or absence of copper sulfide generation at a moment of diagnosis and byproduct.

### FIRST EMBODIMENT

In the present embodiment, insulating oil extracted from an oil-filled transformer is analyzed, and presence or absence of each evaluation parameter from a result of the analysis is used to evaluate a possibility (risk) of copper sulfide generation, so that a degree of risk with regard to occurrence of abnormality in oil-filled electrical equipment is diagnosed. The evaluation parameter includes the five items which are:

(1) presence or absence of DBDS;

(2) presence or absence of DBPC;

(3) presence or absence of an inhibitor for suppressing copper sulfide generation;

(4) presence or absence of oxygen in a space above a surface of insulating oil; and

(5) presence or absence of copper sulfide generation at a moment of diagnosis, or presence or absence of a by product along with copper sulfide generation.

Each item can be detected by means of an existing technology. For example, if measurement equipment such as a gas chromatograph/mass spectrometry device or HPLC (high performance liquid chromatography) is used, quantitative measurement to an extent of 1 ppmw can be performed.

Table 1 is a table for evaluating a possibility (risk) of copper sulfide generation on insulating paper of oil-filled electrical equipment. In Table 1, “copper sulfide or byproduct” corresponds to the item (5) described above.

TABLE 1

| | | DBDS Present | | | | |
|---|---|---|---|---|---|---|
| | | DBPC Present | | | | |
| | | Inhibitor Absent | | | | |
| | | Oxygen | Oxygen | Inhibitor | DBPC | DBDS |
| | | Present | Absent | Present | Absent | Absent |
| Copper | Present | Risk | Risk | Risk | Risk | Risk |
| Sulfide | | Extremely | Middle | Middle | Middle | Middle |
| or | | High | to High | | | |
| Byproduct | Absent | Risk | Risk | Risk | Risk | Risk |
| | | High | Middle | Low | Low | Low |

Based on the analysis result of the five items described above and Table 1, a possibility (risk) of copper sulfide generation can be evaluated highly accurately. Then, a degree of risk with regard to occurrence of abnormality in oil-filled electrical equipment can also be diagnosed highly accurately as can be performed with the risk evaluation.

### EXAMPLE

In relation to the diagnosing method according to the present invention, a result of test for confirming a relationship between the amounts of dibenzyldisulfide (DBDS) and 2,6-di-t-butyl-p-cresol (DBPC) contained in insulating oil and copper sulfide generation on the surface of insulating paper surface and the surface of a copper plate is shown.

Firstly, mineral oil-based insulating oil was prepared for which it was confirmed with IEC62535 that no corrosive sulfur was contained. Next, 300 ppmw (w/w) DBDS was added to this transformer oil to have sample oil A. Further, sample oil B having DBPC of 0.4 weight % (w/w) added to sample oil A was prepared.

Sample oil A and sample oil B were used to conduct a test related to generation of copper sulfide by the method based on IEC62535 of an IEC (International Electrotechnical Commission) standard. For each of sample oil A and sample oil B, sample oil of 15 CC and a copper plate having one layer of craft paper (insulating paper) wound therearound (30 mm×7.5 mm×1.5 mm) are sealed in a bottle having an internal volume of 30 CC, and a silicon rubber stopper is applied, and then heating is performed at 150° C. for 72 hours. Herein, to study a relationship between the amount of oxygen in an atmosphere and copper sulfide generation, air in the bottle is replaced with only nitrogen or with a mixture containing nitrogen and oxygen of 2.5, 5, 10, or 20 volume %.

Table 2 shows a result of evaluation on the state of copper sulfide generation on a surface of a copper plate and a surface of insulating paper after the test. The state of copper sulfide generation was evaluated visually based on the following criterions.

A: No Generation of Copper Sulfide

B: Small Generation at an End of Insulating Paper

C: Generation in a Range Wider than B

D: Generation on a Whole Surface

TABLE 2

| | | Test Atmosphere | | | | |
|---|---|---|---|---|---|---|
| | | | N₂ | N₂ | N₂ | N₂ |
| | | N₂ | 2.5% O₂ | 5% O₂ | 10% O₂ | 20% O₂ |
| Insulating Oil A | Surface of | D | D | D | D | D |
| (DBDS Added) | Copper plate | | | | | |
| | Surface of | A | A | A | A | A |
| | Insulating | | | | | |
| | Paper | | | | | |
| Insulating Oil B | Surface of | D | D | D | D | D |
| (DBDS and DBPC | Copper Plate | | | | | |
| Added) | Surface of | A | B | B | C | D |
| | Insulating | | | | | |
| | Paper | | | | | |

From the result of Table 2, it can be seen that in the case where sample oil B containing both DBDS and DBPC is used, the amount of copper sulfide generation on the insulating paper surface increased as compared to sample oil A containing only DBDS. Further, it can be also understood that the amount of copper sulfide generation on the surface of insulating paper increased as the amount of oxygen in a space above the surface of insulating oil increases.

In other words, it can be determined that the transformer using insulating oil having DBDS and DBPC added has a high risk (a degree of risk with regard to occurrence of abnormality is high). Further, it can be determined that a degree of risk with regard to occurrence of abnormality in the transformer becomes higher as the amount of oxygen in a space above the surface of insulating oil is high.

It is to be understood that the embodiments disclosed herein are only by way of example, and not to be taken by way of limitation. The scope of the present invention is not limited by the description, but rather by the terms of appended claims, and is intended to include any modifications within the scope and meaning equivalent to the terms of claims.

1. A diagnosing method for oil-filled electrical equipment for diagnosing a degree of risk with regard to occurrence of abnormality due to copper sulfide generation in oil-filled electrical equipment, the method comprising:
a first step of detecting specific compounds contained in insulating oil in said oil-filled electrical equipment;
a second step of evaluating a possibility of copper sulfide generation at a dangerous part leading to dielectric breakdown in said oil-filled electrical equipment in accordance with a detection result obtained in said first step; and
a third step of diagnosing a degree of risk with regard to occurrence of abnormality in said oil-filled electrical equipment in accordance with an evaluation result obtained in said second step,
said specific compounds including dibenzyldisulfide and 2,6-di-t-butyl-p-cresol, and
in said second step, a possibility of copper sulfide generation is evaluated taking into consideration presence or absence of oxygen in an atmosphere of said insulating oil.

2. The diagnosing method for oil-filled electrical equipment according to claim 1, wherein said dangerous part is a surface of insulating paper applied to a coil winding wire.

3. The diagnosing method for oil-filled electrical equipment according to claim 1, wherein said specific compounds include a byproduct which is derived when copper sulfide is generated from dibenzyldisulfide.

4. The diagnosing method for oil-filled electrical equipment according to claim 3, wherein said byproduct is at least one kind of compound selected from a group consisting of benzaldehyde, benzyl alcohol, bibenzyl, dibenzyl sulfide, and dibenzyl sulphoxide.

5. The diagnosing method for oil-filled electrical equipment according to claim 1, wherein said specific compounds include an inhibitor for suppressing copper sulfide generation.

6. The diagnosing method for oil-filled electrical equipment according to claim 5, wherein said inhibitor for suppressing copper sulfide generation is a benzotriazole compound.

7. The diagnosing method for oil-filled electrical equipment according to claim 1, wherein
a possibility of copper sulfide generation at a dangerous part leading to dielectric breakdown in said oil-filled electrical equipment is evaluated based on whether each of said specific compounds is detected or not in said first step, and
in said third step, a degree of risk with regard to occurrence of abnormality in said oil-filled electrical equipment is diagnosed as being high when a possibility of copper sulfide generation is evaluated as being high in said second step.

8. (canceled)

9. The diagnosis method for oil-filled electrical equipment according to claim 1, wherein in said second step, a possibility of copper sulfide generation is evaluated further taking into consideration presence or absence of said copper sulfide generation at a moment of diagnosis.
